(19) United States
(12) Patent Application Publication
(10) Pub. No.: US 2022/0177952 A1
(43) Pub. Date: Jun. 9, 2022

(54) GENETIC RISK ANALYSIS FOR POST-TRAUMATIC STRESS DISORDERS AND BEHAVIORAL MANAGEMENT THEREOF

(71) Applicant: Geneus Health, LLC, San Antonio, TX (US)

(72) Inventor: Kenneth Blum, Austin, TX (US)

(73) Assignee: Geneus Health, LLC, San Antonio, TX (US)

(21) Appl. No.: 17/593,556

(22) PCT Filed: Mar. 18, 2020
(86) PCT No.: PCT/US2020/023437

§ 371 Date: Sep. 20, 2021

Related U.S. Application Data
(60) Provisional application No. 62/820,115, filed on Mar. 18, 2019.

(51) Int. Cl. C12Q 1/6827; C12Q 1/6883

(52) U.S. Cl. CPC C12Q 1/6827; C12Q 1/6883; C12Q 2600/106; C12Q 2600/156; C12Q 2600/112

(57) ABSTRACT

Methods for assessing severity index for genetic risks for post-traumatic stress disorders and behavioral management thereof. In some embodiments, the methods provide a risk analysis score (termed a “genetic risk post-traumatic stress disorders score”). The method for behavioral management of those depending upon the individual's genetic risk post-traumatic stress disorders score is termed a precision behavioral management (or “PBM”) protocol.

### RELATED APPLICATIONS

This application claims the benefit of provisional U.S. Patent Application Ser. No. 62/820,115, filed Mar. 18, 2019, entitled Novel Genetic Risk Panel for Post-Traumatic Stress Disorder (PTSD): The Genetic Risk Assessment of Stress Disorder (GRASD™),” which is commonly assigned to the Assignee of the present invention and is hereby incorporated in reference in its entirety for all purposes.

This application is also related to PCT Patent Publ. No. WO/2016/007927, published Jan. 14, 2016, entitled “Genetic Addiction Risk Analysis For RDS Severity Index,” to Blum (“Blum '927 PCT Application”), which is commonly assigned to the Assignee of the present invention and is hereby incorporated in reference in its entirety for all purposes, including the sequence listing provided therein.

### FIELD OF INVENTION

The present invention is directed to methods of assessing severity index for genetic risks for post-traumatic stress disorders and the methods of behavioral management thereof.

### BACKGROUND OF THE INVENTION

### Post-Traumatic Stress Disorder (PTSD)

Post-traumatic stress disorder (PTSD) is a mental health condition that is typically triggered by a terrifying event—either experiencing it or witnessing it. Symptoms may include flashbacks, nightmares, and severe anxiety, as well as uncontrollable thoughts about the event.

It is now widely accepted that individuals who have PTSD also have high co-morbidity of substance use disorder [Bowirrat 2010]. The interrelatedness of reward circuitry and the prefrontal cortices of the brain, and the importance of the core neurotransmitters were not understood when in 1956, the alcoholism concept was introduced by Dr. Jellinek. Without much scientific support, the idea was shocking, and not generally accepted [Jellinek 1960], the discrete functions of serotonin, GABA, dopamine, and acetylcholine were unknown, and endorphins were not even a part of our scientific acumen. However, most addiction scientists at that time agreed that deficiencies or imbalances in brain chemistry—perhaps, genetic in origin—were at least in part the cause of alcoholism.

In 1970, Blum investigated the neurochemical mechanisms of some psychoactive drugs (alcohol and opioids) that had been observed initially in the work of Virginia Davis, Gerald Cohen, Michael Collins, and others as being related to the interface of alcohol- and opioid use disorders [Davis 1970; Hamilton 1978; Collins 1982; Cohen 1970; Blum 1978]. Following many foundational studies from around the world, The Royal Society of Medicine published the Reward Deficiency Syndrome (RDS) concept [Blum 1996]. To date, there have been around 136 RDS articles listed within PubMed. Additionally, The SAGE Encyclopedia of Abnormal and Clinical Psychology included the RDS concept in 2017 [Blum 2017].

### Neuro-Imaging PTSD

Recent neuroimaging research suggests that the self-processing Default Mode Network may be disrupted in many stress-related, psychiatric illnesses, including PTSD [O'Doherty 2017; O'Doherty 2015]. In healthy individuals, the Default Mode Network exhibits the most significant activity during periods of rest, with less activity observed with de-activation during cognitive tasks, e.g., working memory.

Many functional and structural imaging approaches have been developed to study the Default Mode Network which consists of medial temporal regions, lateral parietal cortices, and the medial prefrontal cortex, posterior cingulate cortex/precuneus [Philip 2014]. In a seven-year longitudinal study, Martindale et al. [Martindale 2018] reported that white matter hyperintensities correlated significantly with the intensity of explosion blasts in combat veterans. The white matter hyperintensities did not change with various psychiatric diagnoses and were an independent finding, not explicitly related to PTSD. Also, a preliminary study by Averill et al. [Averill 2017] discovered a negative correlation between cortical thickness in the left rostral middle frontal and left superior temporal regions and combat exposure severity. There was as an interaction between combat exposure severity and PTSD diagnosis in the superior temporal/insular region and a stronger negative correlation between combat exposure severity and cortical thickness in the non-PTSD group. The hippocampus and amygdala are repeatedly implicated in the psychopathology of PTSD. Akiki et al. [Akiki 2017] found that patients with more severe PTSD symptoms showed an indentation (decreased neuronal tissue) in the anterior half of the right hippocampus and an indentation in the dorsal region of the right amygdala (corresponding to the Centro-Medial Amygdala). Moreover, a post hoc analysis that employed stepwise regression suggests that among PTSD symptom clusters, arousal symptoms explained most of the variance in the hippocampal abnormality; whereas re-experiencing traumatic events explained most of the variance in the amygdala abnormality.

It is well known that prolonged stress can have long-lasting effects on cognition [O'Doherty 2015]. Animal models suggest that executive functioning deficits could result from alterations within the mesofrontal circuit. Along these lines, van Wingen and colleagues found that combat stress reduced midbrain activity and integrity and associated with a compromised ability to sustain attention. Long-term follow-up revealed that the functional and structural changes had normalized within 1.5 years. However, a reduction in functional connectivity between the midbrain and prefrontal cortex persisted [van Wingen 2012].

These results demonstrate that combat stress has adverse effects on the human mesofrontal circuit and suggest that these alterations are partially reversible. Such effects impact normal dopaminergic function and reduce the ability to cope with stress. It is shown in magnetic resonance imaging studies that individuals with PTSD, due to prolonged stress, have smaller hippocampi volumes than those without a diagnosis of PTSD [Logue 2018]. Although not well supported in human studies an initial hypothesis involving the mechanism underlying hippocampal alterations in PTSD focused on elevated glucocorticoid levels, in combination with extreme stress as the primary cause. It is noteworthy that Butler et al. [Butler 2018] found that after receiving multimodal, psychological therapy for approximately six weeks, an increase in hippocampal volume and a trend toward an increase in amygdala volume was seen following therapy, with no change observed in the controls.

### Post-Traumatic Stress Disorder is a Subcategory of Reward Deficiency Syndrome

The development of Post-Traumatic stress disorder (PTSD) is a result of complex interplay between environmental and genetic factors. RDS; hypodopaminergia is another neurobiological mechanisms that underlies PTSD, and cross-addictions occur, especially, in psychiatric illness, including PTSD [O'Doherty 2015].

Cocaine use in PTSD is prevalent and associated with negative treatment, health, and societal consequences. Cocaine use disorder appears to increase the risk of PTSD symptoms, especially, in females [Saunders 2015]. Mark Gold's dopamine depletion hypothesis, proposed a vital role for dopamine in the effects of cocaine [Dackis 1985]. Gold et al. suggested that the development of chronic cocaine use disorder (CUD) was due to the euphoric properties of cocaine and followed the acute activation of central dopamine neurons. Overstimulation of these neurons and excessive synaptic metabolism is thought to result in dopamine depletion, which may underlie the dysphoric aspects of cocaine abstinence and cocaine use urges [Dackis 1985]. The neurochemical disruptions caused by cocaine are consistent with the concept of “physical” rather than “psychological” addiction [Noble 1993]. The proposal that followed this research was to treat CUD with dopamine agonist therapy. The powerful dopamine D2 agonist bromocriptine was found to significantly reduce cocaine craving after a single dose [Dackis 1987]. The suggestion was that bromocriptine might be an effective, non-addictive pharmacological treatment for those with CUD and open trials indicated that low-dose bromocriptine might be useful in cocaine detoxification.

Lawford et al. [Lawford 1995] conducted a double-blind study, which administered bromocriptine or placebo to subjects with Alcohol Use Disorder. The most significant improvement in craving and anxiety occurred in the bromocriptine-treated subjects with the DRD2 A1 allele (first associated with severe alcoholism in 1990 by Blum et al. [Blum 1990]), and attrition was highest in the placebo-treated, A1 subjects [Lawford 1995]. However, it is now known that chronic administration of this D2 agonist induces significant down-regulation of D2 receptors, thereby, preventing its clinical use [Bogomolova 2010; Rouillard 1987].

Blum et al. proposed that D2 receptor stimulation could be accomplished with the use of KB220Z [Blum 2008]. His group advocated promoting dopamine release, using milder therapeutic, neuro-nutrient formulations, to increase human mRNA expression causing proliferation of D2 receptors [Blum I 2012]. This proliferation of D2 receptors, in turn, is purported to reduce craving and attenuate stress. Research based on this model has shown that DNA-directed compensatory overexpression of the DRD2 receptors (a form of gene therapy), results in a significant reduction in alcohol craving behavior in alcohol-preferring rodents [Thanos 2005] and self-administration of cocaine [Thanos 2008].

Currently, the most widely accepted approach to treatment for SUD is medication-assisted treatment, which provides an immediate harm reduction strategy to combat substance use disorders.

However, long-term, medication-assisted treatments promote unintentional dopamine down-regulation, and relapse prevention has been poor especially regarding buprenorphine-naloxone combinations. A prudent approach may be biphasic; a short-term blockade, followed by long-term dopaminergic up-regulation, with the goal of enhancing brain dopaminergic function, to target reward deficiency and stress-like, anti-reward symptomatology.

The promotion of long-term, dopaminergic activation by lower potency dopaminergic repletion therapy has been shown clinically to be an effective modality to treat RDS behaviors, including PTSD, substance use disorder, Attention-Deficit/Hyperactivity Disorder (ADHD), obesity and other behaviors that are associated with RDS [Blum 2016]. Increased resting-state functional connectivity and increased neuronal recruitment have also been demonstrated, acutely, with this compound, using fMRI in both animals and humans. It is remarkable that, within 15 minutes (animal) to 60 minutes (human), post-administration of neuro-nutrient therapy, there has been additional dopamine neuronal firing in brain areas involved in reward processing, with possible neuroplasticity as a result [Febo I 2017; Blum 2015]. Moreover, complementary structural and functional neuroimaging techniques used to examine the Default Mode Network could potentially improve understanding of the severity of psychiatric illness symptomatology and provide added validity to the clinical diagnostic process.

The comprehensive role of dopamine as the mesolimbic system neurotransmitter underlying motivational function supports the low potency, dopaminergic repletion therapy concept [Blum II 2012].

### Understanding Genetics of PTSD

David Comings, from the City of Hope, performed the first study to show an association between a reward gene, called the dopamine D2-receptor gene (A1 allele form), with people (military veterans), diagnosed with PTSD [Comings 1996]. In conjunction with Ernest Noble, this same gene form had been shown by Blum's laboratory to associate with severe alcoholism and cause carriers to have 30-40 percent less dopamine D2 receptors in the brain [Blum 1990].

Low dopamine function has been associated with increased risk for PTSD [Noble 1991; Blum III 2012]. It is noteworthy that during combat stress dopamine is released from neurons at 100 times above the resting state. This epigenetic insult then is added to trait hypodopaminergia, (fewer dopamine D2 receptors). There is enough evidence to suggest that susceptibility to PTSD is hereditary. Genetics alone causes about 30% or more of the variance in PTSD. Identical (monozygotic) twins with PTSD, exposed to combat in Vietnam, were associated with an increased risk of their co-twins having PTSD, compared to non-identical (dizygotic) twins [Roy-Byrne 2004].

Additionally, there is evidence that those with smaller hippocampi (a region of the brain involving memory), perhaps due to genetic differences, are more likely to develop PTSD, following traumatic stress. PTSD shares many genetic influences common to other psychiatric disorders. For example, this diagnosis shares 60% of the same genetic variance as panic and generalized anxiety disorders. Alcohol, nicotine, and drugs of abuse share greater than 40% genetic similarity. One study reported that soldiers, whose leukocytes had higher numbers of glucocorticoid receptors (involved in stress response) were more prone to develop PTSD, after experiencing traumatic stress [Blum III 2012; Vaswani 1988].

Genetic antecedents may not tell the whole story since environmental insults or abuse (sexual and verbal) during childhood induce epigenetic changes that impact brain chemistry. Specifically, instead of being caused by differences in the DNA sequence, epigenetic changes are cellular, physiological and behavioral characteristics (phenotypic trait) changes that are caused by external or environmental factors that switch genes on and off. Unfortunately, epigenetic effects can occur for at least two subsequent generations [Szutorisz 2014]. The resultant effects of environmentally-induced, epigenetic changes in the chromatin structure of the DNA have been found, for example, to reduce the function and expression of the dopamine D2 receptor gene, thereby, increasing the likelihood for the development of PTSD.

The take-home message here is that parental abuse in childhood and subsequent exposure to military combat or trauma as an adult may indeed result in PTSD. Genetic polymorphisms are evident in the development of PTSD, due to the regulation of gene expression within the serotoninergic and dopaminergic pathways. The 5-HTTLPR (promoter region of SLC6A4, which encodes the serotonin transporter) is a genetic candidate region that may be responsible for the modulation of emotional responses to traumatic events. Depression and PTSD can be predictable results of the interaction of this variation of the 5HTTLPR gene and stressful life events. The dopaminergic pathway, specifically, the A1 allele coding the type 2 dopaminergic receptor, is associated with severe co-morbidity of PTSD, with the presence of somatic disorders, anxiety, social change, and depression. There is an association between the polymorphism of gene GABRA2 and the occurrence of PTSD. There is an identified interaction between the Val (158)Met polymorphism of the gene coding for catecholamine-o-methyltransferase and number of traumatic events. Other genes include polymorphisms in FKBP5 (a co-chaperone of hsp 90 which binds to the glucocorticoid receptor), which predict PTSD as well, with a gene-by-environment interaction [Auxemery 2012].

Most recently, Zhang et al. [Zhang 2018] investigated the association between PTSD and gene-gene interaction (epistasis) within dopaminergic genes based upon the premise that this information could uncover the genetic basis of dopamine-related PTSD symptomatology and contribute to precision medicine. They found that a statistical analysis of genes identified a DRD2/ANNK1-COMT interaction (r51800497× rs6269), which associates with PTSD diagnosis (_{P interaction}=0.0008055 and P-corrected=0.0169155). Single-variant and haplotype-based subset analyses showed that rs1800497 modulates the association directions of both the rs6269 G allele and the rs6269-rs4633-rs4818-rs4680 haplotype G-C-G-G. The interaction (r51800497× rs6269) was replicated in a young, Chinese female-cohort (32 cases and 581 controls), _{P interaction}=0.01329). The results suggested that rs1800497 is related to the DA (dopamine) receptor D2 density and rs6269-rs4633-rs4818-rs4680 haplotypes affect the catechol O-methyltransferase level and enzyme activity. Thus, the interaction was inferred to be at protein-protein and DA activity level. The genotype combinations of the two SNPs indicate a potential origin of DA homeostasis abnormalities in PTSD development.

PTSD and Evidence-Based Benefits from Pro-Dopamine Regulation

PTSD is thus a psychiatric disorder with a genetic basis [Comings 1996]. The disorder develops from a stress reaction after exposure of a person to physiological and -or psychological trauma, such as sexual assault, warfare, traffic collisions, violence, or other life-threatening events. Symptoms may include re-experiencing the trauma (flashbacks), or nightmares (lucid and non-lucid) related to the events and mental or physical distress induced by trauma-related cues as well as attempts to avoid trauma-related cues. Importantly, symptoms of PTSD include alterations in how a person thinks and feels, like amnesia about the event, fear of relationships, problems with sleeping and concentrating, and being hyper-vigilant (for example startled by loud noises). These symptoms can last for months and even years after the event and PTSD may result in higher suicide risk, long after the initial events [Noble 1991].

Interestingly, not everyone who experiences a traumatic event develops PTSD. However, those who experienced interpersonal trauma, like rape or child abuse, are more likely to develop PTSD, compared to the experience of non-assault-based trauma, such as, accidents and natural disasters. Moreover, after experiencing traumatic, environmental insults (like abuse), victims often experience long-lasting consequences that require treatment. Similarly, young children, who may be unable to process distress can also suffer prolonged adverse sequelae; treatment with a goal of expressing memories through play might help some children experience relief.

According to the Diagnostic and Statistical Manual 5th edition, (DSM-5; American Psychiatric Association 2013), about 3.5% of adults in the US each year have PTSD, and 9% develop it at some point during their lifetime. In the rest of the world, yearly rates are between 0.5% and 1%, although they are much higher in regions of armed conflict. Posttraumatic stress disorder is also more common in women than in men. Outside of the Department of Veterans Affairs clinical environment, the suggestion is that it is necessary for combat soldiers to be able to “handle the trauma” irrespective of any biological propensity to PTSD. It is well-known that some soldiers have, because of this stigma, not been able to seek help. The truth is that many genetic studies from the 1990s until the present day have revealed that specific gene variants (called alleles) predispose some with an inability to handle trauma and stress. One possibly major issue is that because of the shrinking number of people enlisting in the military, and because of poor psychological scores from struggles with substance use, the government has had to reduce the number of applications accepted. The Army is now making it easier for enlistees displaying substance use disorder to obtain acceptance into the military by expanding the use of waivers for recruits with previous marijuana use, bad conduct, and some health problems.

### Can PTSD be Prevented or Treated?

Early access to cognitive behavioral and trauma therapy has been of modest benefit in the treatment of PTSD [Brown 2018]. Also, Critical Incident Stress Management has been suggested as a means of preventing PTSD.

The definition of Critical Incident Stress Management is “an integrated, multi-component continuum of psychological interventions to be provided in the context of acute adversity, trauma, and disaster on an, as needed, basis to appropriate recipient populations.” Critical Incident Stress Management is neither a technique or a treatment for acute stress disorder, PTSD, post-traumatic depression, bereavement, or grief and may cause adverse outcomes.

Interestingly, the World Health Organization recommends against the use of benzodiazepines and antidepressants in those having experienced trauma. Some evidence supports the use of the anti-inflammatory molecule hydrocortisone for prevention in adults. However, there is limited or no evidence supporting other drugs, such as propranolol, escitalopram, temazepam or gabapentin. Indeed, Gabapentin is a drug that stimulates the neurotransmitter GABA that reduces dopamine effects and should not be used to treat PTSD, especially in the long-term [Brown 2012]. However, we are cognizant of the limited short term benefits of Ketamine for depression [Fond 2014].

In response to these recommendations and therapeutic evidence, the use of a pro-dopamine regulator (KB220Z) has been proposed. Studies have been carried out showing that chronic administration of a nutraceutical, KB220Z, eliminated terrifying, lucid nightmares in at least 82% of patients with PTSD and co-morbid ADHD [McLaughlin I 2015; McLaughlin II 2015]. The reduction or elimination of terrifying nightmares was dependent on KB220Z. Voluntary withdrawal of the agent resulted in the reinstatement of the terrifying, non-pleasant nature of the dreams. In most cases, patients reported a gradual, but then complete, amelioration of their long-term, terrifying nightmares (lucid dreams) while taking KB220Z [McLaughlin 2016].

However, it has been shows that in at least four cases the amelioration of nightmares persisted for up to 12 months, after a self-initiated, withdrawal of use of KB220Z [McLaughlin 2016]. These particular cases support the scientific possibility that KB200Z increases dopamine stability as well as functional connectivity between networks (cross-talk between different brain regions) of brain reward circuitry, shown in fMRI studies of both rodents and humans [Blum 2015; Febo I 2017; Febo II 2017]. The increased connectivity volume (recruitment of more dopamine neurons firing in the reward site of the brain) in rodents suggests the induction of epigenetic changes (neuroplastic adaptation), which may be like adaptations involved in human lucid dreaming and nightmares.

Hence, controversies remains about how to modulate dopamine clinically in order to treat and prevent PTSD and various types of addictive disorders. Therefore, a need remains to treat PTSD in soldiers returning to the US after combat. It is essential to find a way to reduce the suffering and trauma experienced by soldiers with untreated PTSD. Reducing the stigma of PTSD by embracing both genetic and epigenetic effects of traumatic stress might influence all people with PTSD to seek out treatment without fear or embarrassment.

### SUMMARY OF THE INVENTION

The present invention is a holistic, therapeutic methods for treating PTSD that includes the a genetic test (such as the Genetic Addiction Risk Score (GARS) test) for genetic risk predisposition and customization of neuronutrient supplementation, based on the GARS test result, coupled with precision behavioral management (PBM). It is believed that gentle D2 receptor stimulation signals a feedback mechanism in the mesolimbic system to increase human mRNA expression causing proliferation of D2 receptors.

In general, in one embodiment, the invention features a method that includes obtaining a biological sample from each of a plurality of subjects. The method further includes that, for each biological sample obtained from the plurality of subjects, performing an allelic analysis on the biological sample to detect the presence of a plurality of pre-determined alleles in the biological sample to identify the severity of a genetic addictive risk for a post-traumatic stress disorder (PTSD). The method further includes identifying a first portion of the subjects in the plurality of subjects that have a high genetic addictive risk of PTSD based upon the allelic analysis of the biological samples. The method further includes administering treatment to subjects in the first portion of subjects. The treatment includes administering neuro-nutrients for dopamine regulation. The treatment further includes administering precision behavioral management directed to achieve dopamine homeostasis.

Implementations of the invention can include one or more of the following features:

The precision behavioral management can be selected from a group consisting of talk therapies, lifestyle measures that promote natural endorphin release, and support groups and systems.

The lifestyle measures that promote natural endorphin release can be selected from a group consisting of diet, mediation, and yoga.

The plurality of subjects can includes people selected from a group of persons consisting of persons who recently enlisted in the military, persons who are enlisting in the military, persons who are trying to enlist in the military, and combinations thereof.

The subjects in the first portion of subjects can receive waiver from the military based upon them receiving the treatment.

The subjects in the first portion of subjects can be assigned non-combat duties in the military based upon the subjects in the first portion of subjects having a high genetic addictive risk of PTSD

The plurality of subjects can include individuals that have already been diagnosed with PTSD.

The individuals can include current and/or former military personnel.

The administering treatments are varied among the subjects in the first portion of subjects based upon the allelic analysis on the biological sample for the subject.

The method can further include identifying a second portion of the subjects in the plurality of subjects that do not have a high genetic addictive risk of PTDS. The method can further include administering treatment to subjects to the second portion of subjects. The treatments administered to the first and second portion of subjects are varied among the subjects in the first and second portion of subjects based upon the allelic analysis on the biological sample for the subject.

The neuro-nutrients can include precursor amino acids.

The neuro-nutrients can be dopamine regulators.

The neuro-nutrients can be regulators for glutamate, dopamine, serotonin, and endorphins.

The method can further include epigenetic testing of at least some of the plurality of subjects.

The method can further include epigenetic testing of at least some of the first portion of the subjects.

The administration treatment to subjects in the first portion of subjects can be varied based upon the epigenetic testing of the subject.

The epigenetic testing can include testing regarding environmental insults and/or abuse during childhood of the subject.

The method can further include identifying a first sub-portion of the first portion of subjects that have a high addictive risk of alcohol and/or substance abuse based upon the allelic analysis of the biological samples. The method can further include varying the administering treatment of the first sub-portion based upon the upon the allelic analysis on the biological sample for the subject.

For the pre-determined alleles being detected in the allelic analysis, each of the alleles in the plurality of alleles can be associated with a gene in a plurality of genes, there can be at least ten genes in the plurality of pre-determined genes, and there can be at least one allele for each of the genes in the plurality of pre-determined genes. The method can further include assigning a count for each of the alleles in the plurality of pre-determined alleles that was detected in the biological subject. The count for the particular allele can be the number of the particular allele detected to be present in the biological sample. The method further can include determining a risk score for the subject based upon the count. The risk score can be the sum of the counts. The identification of the severity of the genetic addictive risk for PTSD can be determined from the count.

The risk score in a first pre-determined range can identify a low genetic addictive risk for PTSD. The risk score in a second pre-determined range can identify a high genetic addictive risk for PTSD.

The second pre-determined range can be 4 and above.

The first pre-determined range can be 0 or 1.

The second pre-determined range can be 7 and above.

The first pre-determined range can be 3 or below.

At least five alleles in the plurality of the pre-determined alleles can include (a) allele G of gene DRD1; (b) allele A1 of gene DRD2; (c) allele S or L of gene HTTLPR; (d) allele G of gene COMT; and (e) allele G of gene OPRM1.

At least eight alleles in the plurality of the pre-determined alleles can be selected from a group consisting of (a) allele G of gene DRD1; (b) allele A1 of gene DRD2; (c) allele C of gene DRD3; (d) allele C of gene DRD4; (e) allele 9R of gene DAT1; (f) allele 7-11R of gene DRD4; (g) allele S or L of gene HTTLPR; (h) allele 4R of gene MAOA; (i) allele G of gene COMT; (j) allele G of gene OPRM1; and (k) allele 181 of gene GABRB3.

The plurality of the pre-determined alleles can include (a) allele G of gene DRD1; (b) allele A1 of gene DRD2; (c) allele C of gene DRD3; (d) allele C of gene DRD4; (e) allele 9R of gene DAT1; (f) allele 7-11R of gene DRD4; (g) allele S or L of gene HTTLPR; (h) allele 4R of gene MAOA; (i) allele G of gene COMT; (j) allele G of gene OPRM1; and (k) allele 181 of gene GABRB3.

The plurality of the pre-determined alleles can be (a) allele G of gene DRD1; (b) allele A1 of gene DRD2; (c) allele C of gene DRD3; (d) allele C of gene DRD4; (e) allele 9R of gene DAT1; (f) allele 7-11R of gene DRD4; (g) allele S or L of gene HTTLPR; (h) allele 4R of gene MAOA; (i) allele G of gene COMT; (j) allele G of gene OPRM1; and (k) allele 181 of gene GABRB3.

### BRIEF DESCRIPTION OF THE DRAWINGS

FIG. 1 is schematic for preventing and treating PTDS.

FIG. 2 is another schematic for preventing and treating PTDS.

### DETAILED DESCRIPTION

The present invention relate to methods of assessing severity index for genetic risks for post-traumatic stress disorders and methods of behavioral management thereof. In some embodiments, the methods provide a risk analysis score (termed a “genetic risk post-traumatic stress disorders score”). The method for behavioral management of those depending upon the individual's genetic risk post-traumatic stress disorders score is termed a precision behavioral management (or “PBM”) protocol.

### PTSD and the Genetic Addiction Risk Score (GARS) Test

There is a need to classify patients at genetic risk for alcohol and drug-seeking behavior before entry to residential and or non-residential chemical dependency and pain programs as well as entry into the military. Concerning the latter, the use of GARS testing (such as disclosed and taught in the Blum '927 PCT Application) has predictive value in identifying individuals faced with military combat who carry high-risk alleles for PTSD. One of the polymorphic variants measured in GARS, specifically the DRD2 A1 allele, has been shown to associate with PTSD and co-morbid substance use disorder. Comings et al. [Comings 1996] studied patients in an addiction treatment unit screened for PTSD, after exposure to severe combat conditions in Vietnam. Of the 24 patients with PTSD, 58.3% carried the D2A1 allele. Of the remaining eight patients, who did not meet PTSD criteria, 12.5% were carriers of the D2A1 allele (p<0.04). Subsequently, in a replication study, using 13 PTSD patients, 61.5% carried the D2A1 allele, 11 patients who did not meet criteria for PTSD did not carry the allele D2A1 (p<0.002). For the combined group, 59.5% with PTSD carried the D2A1 compared to 5.3% of those, who did not have PTSD (p<0.0001) [Comings 1996]. These results suggest that a DRD2 variant in linkage disequilibrium with the D2A1 allele confers an increased risk for PTSD, and the absence of the variant confers relative resistance to PTSD.

Based on an extensive literature review involving thousands of association studies, it has been determined there can be an addiction risk index, based on 11 polymorphisms from 10 genes that are involved in the neurological processing of reward. The GARS score included six SNPs in the DRD1, DRD2, DRD3, DRD4, COMT, and OPRM1 genes; four simple sequence repeats (SSRs) in the DAT1, DRD4, MAOA, and 5HTT transporter genes; and a dinucleotide polymorphism in the GABRA3 gene. Studies by Blum et al., related to GARS [Blum 2018], sought to address genetic risk for alcohol and drug seeking by evaluating whether the combined effect of reward gene polymorphisms contributing to a hypo-dopaminergic trait associated with RDS-related substance abuse risk. Our patient population included 393 poly-drug abusers attending seven independent treatment centers from around the United States. Clinical severity of alcohol and drug use behaviors was assessed using the Addiction Severity Index (ASI-MV). Among those patients who consented to provide DNA via saliva for genotyping, 273 (derived from seven centers) also had ASI phenotypic information. The average age of our analysis sample was 35.3 years of age (S.D. —13.1; range: 18-70) of which 57.8% (n=160) were male and 88.1% (n=244) self-reported their race as Caucasian.

Sequence variations in multiple genes regulating dopaminergic signaling influence risk in an additive manner. For alcohol severity scores, these results strongly suggest that age is a significant covariate. The GARS panel can provide useful information for not only appropriate substance addiction treatment, preliminary screening for high-risk patients in pain clinics, and relapse-prevention, but also the identification of soldiers at high risk for PTSD.

A meta-analysis performed recently on 19 studies that examined with genetic variants in multiple dopaminergic genes and their association with PTSD exhibited inconsistent results [Roy-Byrne 2004]. The combined analysis included 1,752 subjects for rs1800497 in DRD2, 600 for the variable number tandem repeat (VNTR) in the solute carrier family six neurotransmitter transporter (SLC6A3), 1,044 for rs4680 in catechol-O-methyltransferase (COMT), and 394 for rs161115 in dopamine beta-hydroxylase (DBH). The studies meeting eligibility criteria provided data for genetic variants of genes involved in the dopaminergic system included subjects with a diagnosis of PTSD and were case controlled. Using random-effects, meta-analyses under the framework of a generalized linear model (GLM), findings confirmed that the rs1800497 SNP in DRD2 significantly associated with PTSD (OR=1.96, 95% CI; 1.15-3.33; p=0.014). The association of rs1800497 with PTSD remained when only subjects who had experienced combat-related trauma (OR=2.28, 95% CI; 1.08-4.81; p=0.032), or Caucasian subjects alone were the cohort (OR=3.16, 95% CI; 1.34-7.43; p=0.008) [Vaswani 1988]. Furthermore, the 30-UTR VNTR in SLC6A3 also showed significant association with PTSD (OR=1.62, 95% CI; 1.12-2.35; P=0.010).

The meta-analysis did not find an association with PTSD for the COMT Val 158Met rs4680 allele (OR=0.91, 95% CI: 0.63-1.30; P=0.595) or the DBH homozygous TT rs161115 (OR=1.55, 95% CI: 0.39-6.20; P=0.536). However, due to the limited sample size examination of the association of rs161115 with PTSD, requires larger additional studies for validation. Importantly, information about the severity and type of traumatic events across studies in this meta-analysis were variable. This lack of data and heterogeneity regarding trauma exposures may explain, in part, the lack of an association between rs4680 in COMT with PTSD.

TABLE 1 is a list of with genetic variants from multiple dopaminergic genes used in GARS and their association with PTSD including the phenotype, the gene, the association, and the citation.

TABLE 1
Polymorphic Reward Genes in GARS to associate with PTSD and related behaviors

| | | | |
|---|---|---|---|
| Polymorphism | Study Findings | References | Comments |

DRD1:

| | | | |
|---|---|---|---|
| rs5326-A allele at | Poor strategic | Tsang 2015 | Poor logic or poor |
| the promoter | Planning | | cognitive |
| region of | Poor logic or | | strategic planning leads |
| dopamine receptor | judgement | | to PTSD |
| D1 (DRD1) locus | | | |
| CHR5 | | | |
| rs4532 | deficit in attention, | Minichino 2017 | Poor focus and lack of |
| | avolition | | goals can exacerbate |
| | | | PTSD behaviors |
| rs4532 | Impulse Control | Zainal 2015 | Impulse control |
| | Disorder | | underpins |
| | | | PTSD behaviors |
| rs4532 | Association with | Prasad 2013 | Alcoholism is highly |
| | Alcohol dependence | | comorbid with PTSD |
| rs4532 | Reduced post heroin | Zhu 2013 | important diagnostic |
| | Dependence pleasure | | feature of PTSD is |
| | | | anhedonia |
| rs4532 | Ubanicity upbringing | Reed 2018 | Ubanicity in DRD1 |
| | alters PFC function | | polymorphic an |
| | | | antecedent for |
| | | | Psychiatric disorders |

DRD2:

| | | | |
|---|---|---|---|
| rs1800497 | linkage | Comings 1996 | A1 allele confers high |
| | disequilibrium A1 | | risk for PTSD and |
| | allele | | A2 allele protects |
| rs1800497 | reduces dopamine | Zhang 2018 | Interaction with COMT |
| | homeostasis in PTSD | | confers PTSD risk |
| rs1800497 | significantly | Li 2016 | Based on a meta-analysis |
| | associates with PTSD | | |
| | risk | | |
| rs12364283 | associated high risk | Nelson 2014 | War Veterans Cohort |
| | for PTSD in | | |
| | psychostimulant | | |
| | abuse | | |
| rs1800497 | A1 allele magnifies | Hemmings 2013 | South African Cohort |
| | PTSD severity | | |
| single nucleotide | significant | Voisey 2009 | War Veterans Cohort |
| olymorphism | association PTSD risk | | |
| (SNP) (957C > T) | | | |
| rs1800497 | carriers of A1 allele | Lawford 2006 | Combat Veterans with |
| | increased Mississippi | | DRD2 show severe |
| | Scale for Combat | | PTSD symptoms. |
| | PTSD score | | |

DRD3:

| | | | |
|---|---|---|---|
| rs6280 | aberrant decision- | Rajan 2018 | DRD3 polymorphism |
| | making | | will exacerbates poor |
| | | | decision-making in |
| | | | PTSD |
| rs6280 | significantly | Oporto 2018 | There is a high |
| | associated with | | comorbidity of Bruxism |
| | Bruxism | | and PTSD |
| rs6280 | carrying the Ser9Gly | Zhao 2016 | People with PTSD have |
| | variant associates | | an inability to for social |
| | with poor social | | conformity style |
| | conformity | | |
| rs6280 | significant | Kang 2014 | High co-morbidity of |
| | association with | | AD and PTSD |
| | alcohol dependence | | |
| | (AD) | | |
| rs6280 | reduced executive | Bombin 2008 | Poor decision-making is |
| | function | | comorbid with PTSD |

DRD4:

| | | | |
|---|---|---|---|
| 48 base repeat | Intense PTSD | Dragan 2009 | Flood Victims Cohort |
| VNTR (Chr11 | Symptoms | | |
| exon3) | | | |
| 7 R & 8R | | | |
| 48 base repeat | Low Cortisol | Armbruster 2009 | Inability to respond to |
| VNTR ( Chr 11 | | Response | stressful events |
| exon3) 7R | | | |
| 48 base repeat | Low resilience to | Azadmarzabadi | DRD4 7R associated |
| | stress | 2018 | with personality |
| | | | anxiety, depression, |
| | | | intelligence |
| 48 base repeat | High Life Stress | Brody 2018 | Life Stress X DRD4 & R |
| VNTR(Chr11 | | | carriers increase drug |
| exon3)7R + R | | | use in African |
| | | | Americans |
| 48 base repeat | Association with | Bakermans- | Carriers of the DRD4 |
| VNTR (Chr 11 exon | childhood loss of | Kranenburg | 7R have a lower ability |
| 3) 7R | trauma | 2011 | to deal with parental |
| | | | problems which |
| | | | increases children's risk |
| | | | for later |
| | | | psychopathology |
| | | | including PTSD |

DA1:

| | | | |
|---|---|---|---|
| 40 bases repeat | Carriers of 9R DAT1 | Segman 2002 | Dopamine genetics |
| VNTR (hr5, exon15) | associates wit PTSD | | contribute to PTSD in |
| 9R | | | Trauma Survivors |
| 40 bases repeat | Children that carry | Drury 2013 | Understanding this |
| VNTR(Chr5, exon | the 9 R and the C | | genetic antecedent |
| 15) 9R (rs28363170) | allele have high | | provides future |
| and C allele of | PTSD following | | personalized epigenetic |
| rs27072 | trauma than those | | repair |
| | that do not carry this | | |
| | haplotype | | |
| 40 bases repeat 9R | anxiety traits and n | Hünnerkopf 2007 | interaction with harm |
| | | | avoidance and |
| | | | Neuroticism |
| 40 bases repeat 9R | PTSD symptoms | Li 2016 | Meta-analysis on |
| | | | 19 studies |
| 40 bases repeat 9R | PTSD symptoms | Valente 2011 | Post Violent Urban Victims |

COMT:

| | | | |
|---|---|---|---|
| Val^{158/108}Met rs4680 | One or two Met | Mestrovic 2018 | Val/Val carriers |
| Chr22 | copies are protective | | performed worst with s |
| G allele | against dysfunctional | | high delay in recall in |
| | working memory | | PTSD |
| Val¹⁵⁸Met | One or two copies of | Deslauriers 2018 | Combat Marines with |
| | Met protects induces | | PTSD |
| | fear inhibition | | |
| Val¹⁵⁸Met | One or two copies of | Winkler 2017 | Val variant associates |
| | Met protects against | | with poor Global |
| | impaired Global | | Functional Outcome |
| | Functional Outcome | | |
| Val¹⁵⁸Met | Trauma in pre- | Humphreys 2014 | importance of |
| | school varies with | | moderating influence of |
| | race in African | | race on genotype |
| | Americans Met/Met | | |
| | higher PTSD | | |
| | symptoms but in EU | | |
| | Caucasian Val/Val | | |
| | higher PTSD. | | |
| Val¹⁵⁸Val | Homozygote Val/Val | Clark 2014 | Iraq Combat Veterans |
| | compared to Val/Met | | |
| | Showed increased | | |
| | PTSD symptoms in | | |
| | face of combat | | |
| | trauma | | |
| Val¹⁵⁸Met Rs 4680 | Carriers of | Boscarino 2012 | FKBP5, CHRNA5, |
| | rs4680SNP had high | | CRHRland COMT |
| | PTSD symptoms as | | contribute PTSD |
| | part of a gene risk | | |
| | count of three other | | |
| | genes | | |

MOA-A:

| | | | |
|---|---|---|---|
| 30 bases repeat | Maltreatment in | Zhang 2017 | The aggression |
| VNTR Chr X | childhood in carriers | | phenotype was |
| Promoter 3.5R, 4R | MOA-A VNTR had | | magnified with the |
| | highest PTSD | | serotonin risk genotype |
| | aggressive type | | |
| | symptoms | | |
| MOA-A H | In females not men | Verhoeven 2012 | The aggression in |
| 3.5R and 4R | the MOA-A-H | | females with the |
| | genotype showed | | high activity genotype |
| | high aggression | | occurs when sadness is |
| | | | present. |
| | | | Opposite evidence for |
| | | | MOA-A L |
| | | | Higher amounts of |
| | | | available Dopamine |
| | | | could lead to increased |
| | | | especially in males |
| | | | [Frazzetto 2007] |

5-HTTLPR (SLC6A4):

| | | | |
|---|---|---|---|
| 43 bases repeat | Through Gene wide | Mehta 2018 | Australian and US |
| INDEL/VNTR | studies the serotonin | | Marine cohort |
| Chr 17 | transporter risk | | |
| Risk alleles LG. S | alleles associated | | |
| | with PTSD | | |
| Rs25531 and risk | Increased risk for | Tian 2015 | Both 5-HTTLR and %- |
| alleles | PTSD in | | HTTVNTR |
| | Earthquake survivors | | polymorphisms |
| | | | associated with PTSD |
| Rs25531 and risk | increases odds of 1.5 | Liu 2015 | Combat exposure cohort |
| alleles | of Combat Vets of | | |
| | having PTSD | | |
| Triallelic 5-HTTLPR | Homozygote SS | Walsh 2014 | African American |
| | genotype protects | | Cohort childhood |
| | against PTSD | | emotional abuse |
| | re-experience of | | |
| | trauma | | |
| Triallelic 5-HTTLPR | Homozygote SS | Gressier 2013 | Meta-analysis mixed |
| | genotype increased | | ethnic group |
| | risk of PTSD in high | | |
| | trauma | | |
| Triallelic 5-HTTLPR | Carriers of one or | Xie 2012 | In a large mixed ethnic |
| | two copies | | group the S genotype |
| | S genotype confer | | associated with anxiety |
| | increased risk of | | and depression |
| | PTSD in childhood | | |
| | adversity | | |
| rs16965628 | modulated task- | Morey 2011 | Combat related trauma |
| (SLC6A4) | related ventrolateral | | |
| | PFC activation in | | |
| | PTSD | | |
| short (S)/long (L) | additive excess risk | Grabe 2009 | 60% of all L(A) allele |
| of 5-HTTLPR (re | for frequent trauma | | carriers exposed to 3 or |
| 4795541) | in the L(A)/L(A) | | more traumas developed |
| | genotype | | PTSD |

Mu-Opioid Receptor (OPRM1):

| | | | |
|---|---|---|---|
| rs1799971 Chr6 | G carriers increase in | Carver 2016 | Enhanced sensitivity to |
| Risk allele G | anger proneness | | negative environments |
| | hostility, ego, | | loads onto PTSD |
| | negative feelings | | |
| rs1799971 | G carriers compared | Slavich 2014 | Adolescent Cohort |
| | to A become more | | |
| | depressed when | | |
| | faced with social | | |
| | rejection | | |

GABRB3:

| | | | |
|---|---|---|---|
| Alpha 3 Chr15 | increased somatic | Feusner 2001 | Carriers non G allele in |
| (DNR) | symptoms, anxiety, | | PTSD patients show |
| Risk allele 181 ( non | insomnia, social | | high co-morbidity |
| G1) | dysfunction, | | |
| | depression | | |

### Processes for Preventing and Treating PTSD

Generally, the process for preventing and treating PTSD is a combination of characterizing genotype-phenotype relationships of the individual and them implementing precision behavioral management (that can include putative pro-dopamine regulation accompanied by interventions like mindfulness and cognitive behavioral therapy as well trauma therapy).

In some embodiments, enlistees for military duty are pre-tested for risk of PTSD with the genetic testing so that that the process can be pro-active. Indeed, even before combat, military personnel with a childhood background of violence (or with a familial susceptibility risk) would benefit from being genotyped for high-risk alleles (DNA variants). This process will assist in identifying potential military candidates who would be less well-suited for combat than those without high-risk alleles.

Furthermore, safe methods to treat individuals already exposed to combat and known to have PTSD can be treated with the present invention. Since hypodopaminergic function in the brain's reward circuitry due to gene polymorphisms (variations) is known to increase substance use disorder in individuals with PTSD, after characterizing genotype-phenotype relationships, this information can be utilized to administer precision, pro-dopamine regulators (such as KB220PAM), to affect the epigenetic expression (mRNA) to overcome this deficiency.

In this way, soldiers would be less vulnerable to traumatic stress and more likely to be able to “handle trauma,” which is not based solely on non-biological stigimatization. While the genetic testing alone is informative, when used to guide precision pro-dopamine regulators and other treatments, this will assist in increasing military personnel's health and increase the number of acceptable applicants with high risks for both PTSD and substance use disorder, due to subsequent amelioration of these co-morbid pre-conditions.

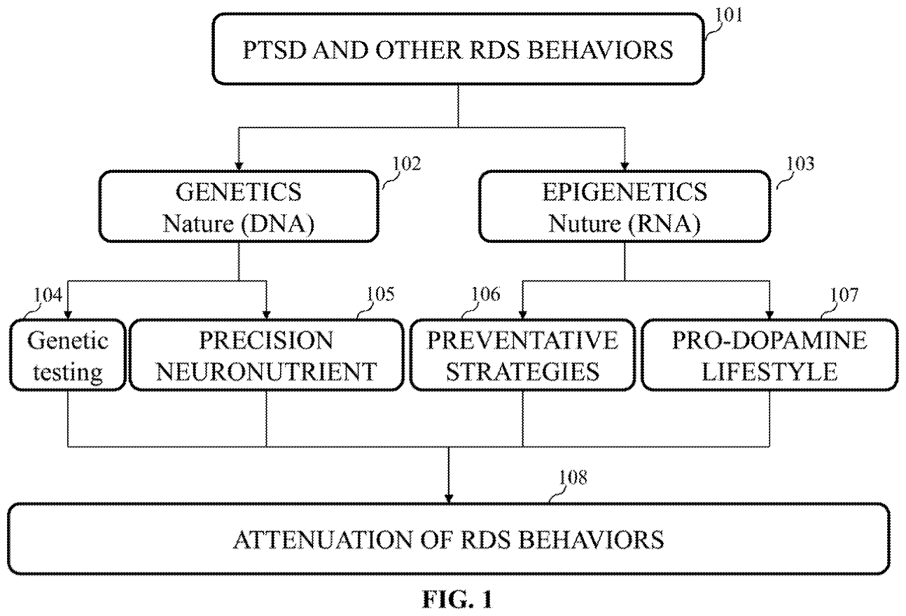

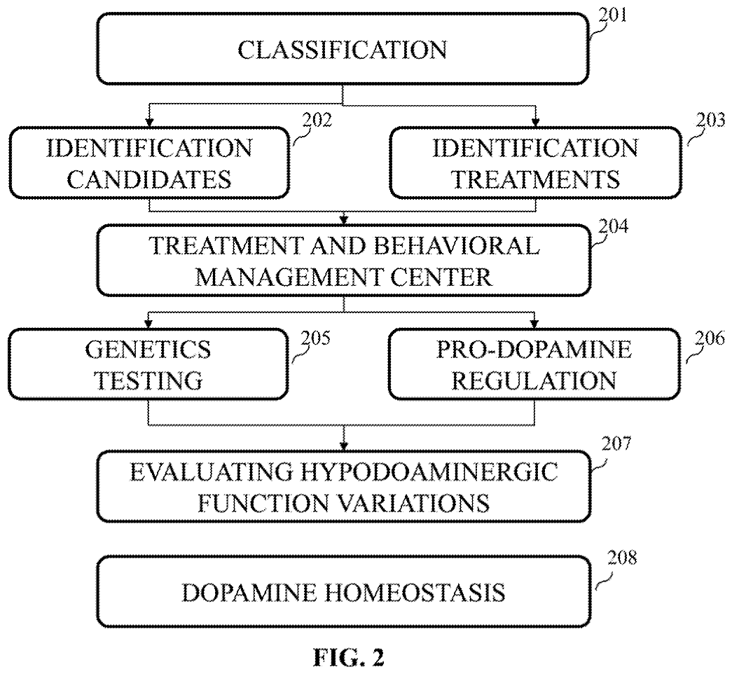

FIGS. 1-2 are schematics processes to prevent and treat PTSD.

FIG. 1. shows that problem 101 to be addressed is PTSD and other reward deficiency syndrome (RDS) behaviors that accompany PTSD. Problem 101 is can be separated into two parts namely genetics 102, i.e., nature (DNA) and epigenetics 103, i.e., nuture (RNA). Epigenetics 103 includes environmental insults or abuse (sexual, verbal, etc.) during childhood.

For genetics 102, solutions include genetic testing 104 (such as the GARS testing disclosed and taught in the Blum '927 PCT Application) and administering precision neuro-nutrients 105 based upon the genetic testing. For epigenetics 103, the solutions include preventative strategies 106 and a pro-dopamine lifestyle 107. These solutions yield results 108 of attenuating RDS behaviors, including PTSD and related behaviors, such as substance and non-substance addictions. For example, the GARS testing to encompass the testing shown in TABLE 8 of the Blum '927 PCT Application, in which the analysis was performed by performing an allelic analysis of the following alleles: (a) allele G of gene DRD1; (b) allele A1 of gene DRD2; (c) allele C of gene DRD3; (d) allele C of gene DRD4; (e) allele 9R of gene DAT1; (f) allele 7-11R of gene DRD4; (g) allele S or L of gene HTTLPR; (h) allele 4R of gene MAOA; (i) allele G of gene COMT; (j) allele G of gene OPRM1; and (k) allele 181 of gene GABRB3.

The risk allele count is based upon the positive indications of the alleles in the panel tested. The risk allele count can be segregated into low, moderate, and high ranges for genetic addictive risk for PTSD. For example, for PTSD, the high range can be 4 and above, and the low range can be 0 or 1. Further, for example, for PTSD, the high range can be 7 and above, and the low range can be 3 or below. Moreover, the risk allele count can also be utilized for other addictive behaviors that accompanying PTSD, such as substance abuse. For, example, a genetic addictive risk score of 7 and above indicates a high genetic addictive risk for substance abuse of alcohol. Further, for example, a genetic addictive risk score of 4 and above indicates a high genetic addictive risk for substance abuse of drugs, including opiates.

More globally, the genes panels for PTSD can include genes selected from the following group: DRD1-5, DAT1, Delta, Mu, Kappa, Sigma and Gamma receptors, all known serotonin receptors, all known Cannibinoid receptors; Glutaminergic receptors, Cholinergic receptors, Gabanergic receptors, serotonin transporters, MOA-A, and MOA-B, COMT, NETO2, GLUK5, FKBP51, TEF, NR3C1, CRF1, GRIA4, BDNF, Per1/Per2, FGF21, MAPT, CACNA1C, AGO2, DCR1, ADCYAP1R1, GR-1F, IL1B, NOS1AP, NOS1, HDAC4. GAT1, Notchl, NLGN1, ZNRD1-AS1, NF-κB, CCK, ALOX, angiotensin II receptor type 1a (AT1a R), SLC6A2, apolipoprotein E (APOE) c4, SNCA, PRTFDC1, TPH2), SLC1A1, Grinl), ADRB2, OXTR, CD38 and AVPR1a, WWC1 (KIBRA), Bcl-2, PACAP, PAC1, FAAH, TPH1, PP1, TACR1, NT-3 (TgNTRK3), DBH, and glyoxalase 1.

FIG. 2 shows that the process can start with a classification step 201 in which individuals at high genetic risk or with a familial susceptibility for PTSD are classified. For individuals who are entering enlistees for military duty, this can include pre-testing and genotype analysis for high risk alleles (DNA-variants) of PTSD, such as by using the GARS testing shown in the Blum '927 PCT Application.

This classification step will assist in the identification steps of steps 202 and 203. In step 202, military candidates can be identified who are less suited for combat based upon having a high number of high-risk alleles. In step 203, the identification assists in identifying and classifying individuals already exposed to combat and known to have PTSD and finding safe and effective treatments for them.

In step 204, those individuals that have been identified can then be sent to a treatment and behavioral management center. These individuals have not necessarily been exposed to trauma already that would cause PTSD or related RDS, but rather, this can be done for preventive actions.

Indeed, early diagnosis through genetic testing (including pharmacogenetics and pharmacogenomics), treatment with pro-dopamine regulation as a goal, and appropriate urine drug screening could conceivably reduce stress, craving, and relapse and enhance well-being in the recovery community. These actions could lead to eventual attenuation of PTSD, because of early identification.

In step 205, further genetic testing can be done. Moreover, in some cases the testing may not have been performed by an individual being sent to the center. For instance, there could be individuals who were sent to the center because they had already showed signs of PTSD or because they had a familial susceptibility risk. Again, this genetic testing can be the GARS testing shown in Blum '927 PCT Application.

In step 206, a precise ingredient based-dopamine regulation, such as KB220PAM, can be used that is genetically tied to formulae matched to the polymorphic genes specific to the individual.

In step 207, further evaluation is performed to evaluation the hypodopaminergic function due to gene polymorphism (variations) caused by substance use disorders in the individuals.

In step 208, dopamine homeostatis is sought to be achieved by customized precision behavioral management (i.e., neuro-nutrient supplement). This can be tailored to effect the epigenetic expression (RNA) to overcome this deficiency. Step 208 can include (a) talk therapies, (b) lifestyle measures that promote natural endorphin release (like diet and mediation), and (c) support groups and systems.

Step 208 can be accompanied by interventions (like mindfulness and biosensor tracking). A program that teaches pro-dopamine lifestyle and uses urine drug screens like the Comprehensive Analysis of Reported Drugs (CARD) to monitor outcomes and serve as a basis for therapeutic interactions can be utilized for addictions that typically accompany PTSD.

A pro-dopamine lifestyle, with gentle, prolonged D2 agonist therapy to compensate for DNA polymorphisms, can promote positive epigenetic effects that can be transferred from generation to generation [McLaughlin 2013; Thanos 2016]. Holistic modalities like low glycemic index diet; mindfulness training, neurofeedback, yoga, and meditation are known to naturally release neuronal dopamine [Blum 2010; Kjaer 2002], and, supported by the 12-step fellowship, might induce feelings of well-being and reduce craving and relapse.

There was as an interaction between combat exposure severity and PTSD diagnosis in the superior temporal/insular region and a stronger negative correlation between combat exposure severity and cortical thickness in the non-PTSD group. The hippocampus (memory recall) and amygdala (fear) are repeatedly implicated in the psychopathology of PTSD. Akiki et al. [Akiki 2017] found that patients with more severe PTSD symptoms showed an indentation (decreased neuronal tissue) in the anterior half of the right hippocampus and an indentation in the dorsal region of the right amygdala (corresponding to the Centro-Medial Amygdala). In addition, a post hoc analysis that employed stepwise regression suggests that among PTSD symptom clusters, arousal symptoms explained most of the variance in the hippocampal abnormality; whereas re-experiencing traumatic events explained most of the variance in the amygdala abnormality. These results suggest that therapeutic targets to attenuate PTSD symptoms should be directed to desenstization of hippocampus recall and attenuation of stress related fear steming from the Centro-Medial Amygdala.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. The scope of protection is not limited by the description set out above, but is only limited by the claims which follow, that scope including all equivalents of the subject matter of the claims.

The disclosures of all patents, patent applications, and publications cited herein are hereby incorporated herein by reference in their entirety, to the extent that they provide exemplary, procedural, or other details supplementary to those set forth herein.

Amounts and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of approximately 1 to approximately 4.5 should be interpreted to include not only the explicitly recited limits of 1 to approximately 4.5, but also to include individual numerals such as 2, 3, 4, and sub-ranges such as 1 to 3, 2 to 4, etc. The same principle applies to ranges reciting only one numerical value, such as “less than approximately 4.5,” which should be interpreted to include all of the above-recited values and ranges. Further, such an interpretation should apply regardless of the breadth of the range or the characteristic being described.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about” and “substantially” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “substantially perpendicular” and “substantially parallel” is meant to encompass variations of in some embodiments within ±10° of the perpendicular and parallel directions, respectively, in some embodiments within ±5° of the perpendicular and parallel directions, respectively, in some embodiments within ±1° of the perpendicular and parallel directions, respectively, and in some embodiments within ±0.5° of the perpendicular and parallel directions, respectively.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

### REFERENCES

Akiki, T. J., et al., “The Association of PTSD Symptom Severity with Localized Hippocampus and Amygdala Abnormalities,” Chronic Stress (Thousand Oaks, Calif.), 2017; 1 (“Akiki 2017”).
Armbruster, et al., Behav Neuroci, 2009; 123(6):1288-95 (“Armbuster 2009”).
Auxemery, Y., “Posttraumatic stress disorder (PTSD) as a consequence of the interaction between an individual genetic susceptibility, a traumatogenic event and a social context,” Encephale, 2012; 38(5):373-80 (“Auxemery 2012”).
Averill, L. A., et al., “Combat Exposure Severity is Associated with Reduced Cortical Thickness in Combat Veterans: A Preliminary Report,” Chronic Stress (Thousand Oaks, Calif.), 2017; 1 (“Averill 2017”).
Azadmarzabadi, et al., Psychgeriactrics, 2018; 18(3): 190-201 (“Azadmarzabadi 2018”).
Bakermans-Kranenburg, et al., Attch Hum Dev., 2011; 13(3):253-69 (“Bakermans-Kranenburg 2011”).
Blum, K., et al., “Genetic addiction risk score (GARS), a predictor of vulnerability to opioid dependence,” Frontiers in Bioscience (Elite edition), 2018; 10:175-96 (“Blum 2018”).
Blum, K., Reward Deficiency Syndrome. In: Wenzel A, editor, The SAGE Encyclopedia of Abnormal and Clinical Psychology University of Pennsylvania School of Medicine, USA: Sage Publications, Inc; 2017, p. 4 200 (“Blum 2017”).
Blum, K., et al., “Fifty Years in the Development of a Glutaminergic-Dopaminergic Optimization Complex (KB220) to Balance Brain Reward Circuitry in Reward Deficiency Syndrome: A Pictorial,” Austin Addiction Sciences, 2016; 1(2) (“Blum 2016”).
Blum, K., et al., “rsfMRT effects of KB220Z on neural pathways in reward circuitry of abstinent genotyped heroin addicts,” Postgrad Med., 2015; 127(2):232-41 (“Blum 2015”).
Blum, K., “Neurogenetics and Nutrigenomics of Neuro-Nutrient Therapy for Reward Deficiency Syndrome (RDS): Clinical Ramifications as a Function of Molecular Neurobiological Mechanisms,” J Addict Res Ther., 2012; 3(5):139 (“Blum I 2012”).
Blum, K., et al., “‘Liking’ and ‘wanting’ linked to Reward Deficiency Syndrome (RDS): hypothesizing differential responsivity in brain reward circuitry,” Curr Pharm Des., 2012; 18(1):113-8 (“Blum II 2012”).
Blum, K., et al., “Diagnosis and Healing In Veterans Suspected of Suffering from Post-Traumatic Stress Disorder (PTSD) Using Reward Gene Testing and Reward Circuitry Natural Dopaminergic Activation,” J Genet Syndr Gene Ther 2012; 3(3):1000116 (“Blum III 2012”).
Blum, K., et al., “Overcoming qEEG abnormalities and reward gene deficits during protracted abstinence in male psychostimulant and polydrug abusers utilizing putative dopamine D(2) agonist therapy: part 2,” Postgrad Med., 2010; 122(6):214-26 (“Blum 2010”).
Blum, K., et al., “Activation instead of blocking mesolimbic dopaminergic reward circuitry is a preferred modality in the long term treatment of reward deficiency syndrome (RDS): a commentary,” Theor Biol Med Model, 2008; 5:24 (“Blum 2008”).
Blum, K., et al., “The D2 dopamine receptor gene as a determinant of reward deficiency syndrome,” J R Soc Med., 1996; 89(7):396-400 (“Blum 1996”).
Blum, K., et al., “Allelic association of human dopamine D2 receptor gene in alcoholism, Jama., 1990; 263 (15):2055-60 (“Blum 1990”).
Blum, K., et al., “Putative role of isoquinoline alkaloids in alcoholism: a link to opiates,” Alcohol Clin Exp Res., 1978; 2(2):113-20 (“Blum 1978”).
Bogomolova, E. V., et al., “Dopamine down-regulates activity of alkaline phosphatase in Drosophila: the role of D2-like receptors,” Journal of Insect Physiology, 2010; 56(9):1155-9 (“Bogomolova 2010”).
Bombin, et al., Am J Med Genet B Neuropsychiatr Genet., 2008; 147B(6):873-9 (“Bombin”).
Boscarino, et al., Neuropsychiatric Dis. Treat, 2012; 8:131-9 (“Boscarino 2012”).
Bowirrat, A., et al., “Neuro-psychopharmacogenetics and Neurological Antecedents of Posttraumatic Stress Disorder: Unlocking the Mysteries of Resilience and Vulnerability,” Curr Neuropharmacol., 2010; 8(4):335-58 (“Bowirrat 2010”).
Brody, et al., Dev Psychopathol., 2018; 24(3):941-51 (“Brody 2018”).
Brown, L. A., et al., “Does CBT for anxiety-related disorders alter suicidal ideation? Findings from a naturalistic sample,” Journal of Anxiety Disorders, 2018; 59:10-6 (“Brown 2018”).
Butler, O., et al., “Hippocampal gray matter increases following multimodal psychological treatment for combat-related post-traumatic stress disorder,” Brain and Behavior, 2018; 8(5):e00956 (“Butler 2018”).
Carver, et al., Soc Neurosci, 2016; 11(5):515-24 (“Carver 2016”).
Clark, et al., J Psychiatric Res., 2014; 47(12): 1849-56 (“Clark 2014”).
Cohen, G., et al., “Alkaloids from catecholamines in adrenal tissue: possible role in alcoholism,” Science, 1970; 167(3926): 1749-51 (“Cohen 1970”).
Collins, M. A., et al., “Attraction to ethanol solutions in mice: induction by a tetrahydroisoquinoline derivative of L-DOPA,” Subst Alcohol Actions Misuse, 1982; 3(5):299-302 (“Collins 1982”).
Comings, D. E., et al., “Dopamine D2 receptor (DRD2) gene and susceptibility to posttraumatic stress disorder: a study and replication,” Biol Psychiatry, 1996; 40(5):368-72 (“Comings 1996”).
Dackis, C. A., et al., “Climko R. Single-dose bromocriptine reverses cocaine craving,” Psychiatry Research, 1987; 20(4):261-4 (“Dackis 1987”).
Dackis, C. A., et al., “New concepts in cocaine addiction: the dopamine depletion hypothesis,” Neuroscience and Biobehavioral Reviews, 1985; 9(3):469-77 (“Dackis 1985”).
Davis, V. E., et al., “Alcohol addiction and tetrahydropapaveroline,” Science, 1970; 169(3950): 1105-6 (“Davis 1970”).
Deslauriers, et al., Depress Anxiety, 2018; 35(1):32-42 (“Deslauriers 2018”).
Dragan, et al., Anxiety Stress Coping, 2009; 22(5):483-95 (“Dragan 2009”).
Drury, et al., J Child Adolesc Psychopharmacology, 2013; 23(4):236-43 (“Drury 2013”).
Febo, M., et al., “Dopamine homeostasis: brain functional connectivity in reward deficiency syndrome,” Frontiers in Bioscience (Landmark edition), 2017; 22:669-91 (“Febo I 2017”).
Febo, M., et al., “Enhanced functional connectivity and volume between cognitive and reward centers of naive rodent brain produced by pro-dopaminergic agent KB220Z,” PLoS One, 2017; 12(4): e0174774 (“Febo II 2017”).
Feusner, et al., Psychiatry Research, 2001; 104(2): 109-17 (“Feusner 2001”).
Fond, G., et al., “Ketamine administration in depressive disorders: a systematic review and meta-analysis,” Psychopharmacology (Berl), 2014; 231(18):3663-76 (“Fond 2014”).
Frazzetto, et al., Plosone, 2007; 2(5):e486 (“Frazzetto 2007”).
Grabe, et al., Am J Psychiatry, 2009; 166(8):926-33 (“Grabe 2009”).
Gressier, et al., J Trauma Stress, 2013; 26(6):645-53 (“Gressier 2013”).
Hamilton, M. G., et al., “Identification of an isoquinoline alkaloid after chronic exposure to ethanol,” Alcohol Clin Exp Res., 1978; 2(2):133-7 (“Hamilton 1978”).
Hemmings, et al., Prog Neuropyschopharmacology Biol Psychiatry, 2013; 40:273-80 (“Hemmings 2013”).
Humphreys, et al., J Child Adolesc Psychopharmacology, 2014; 24(8):454-7 (“Humphreys 2014”).
Hünnerkopf, et al., Neuropsychopharmacology, 2007; 32(12):2552-60 (“Hunnerkopf 2007”).
Jellinek, E., The Disease Concept of Alcoholism. Italy: New Heaven: College and University Press; 1960 (“Jellinek 1960”).
Kang, et al., Neuropsychobiolog, 2014; 69(3):140-6 (“Kang 2014”).
Kjaer, T. W., et al., “Increased dopamine tone during meditation-induced change of consciousness,” Brain Research Cognitive Brain Research, 2002; 13 (2): 255-9 (“Kjaer 2002”).
Lawford, et al., Eur psychiatry, 2006; 21(3):180-5 (“Lawford 2006”).
Lawford, B. R., et al., “Bromocriptine in the treatment of alcoholics with the D2 dopamine receptor A1 allele,” Nat Med., 1995; 1(4):337-41 (“Lawford 1995”).
Li, et al., Medicine, 2016; 95 (11):e3074 (“Li 2016”).
Liu, et al., Plosone, 2015; 10(3):e0119998 (“Liu 2015”).
Logue, M. W., et al., “Smaller Hippocampal Volume in Posttraumatic Stress Disorder: A Multisite ENIGMA-PGC Study: Subcortical Volumetry Results From Posttraumatic Stress Disorder Consortia,” Biol Psychiatry., 2018; 83(3):244-53 (“Logue 2018”).
Martindale, S. L., et al., “Longitudinal changes in neuroimaging and neuropsychiatric status of post-deployment veterans: a CENC pilot study,” Brain Injury, 2018; 32(10):1208-16 (“Martindale 2018”).
McLaughlin, T., et al., “KB220Z™ a Pro-Dopamine Regulator Associated with the Protracted, Alleviation of Terrifying Lucid Dreams. Can We Infer Neuroplasticity-induced Changes in the Reward Circuit?” J Reward Defic Syndr Addict Sci., 2016; 2(1):3-13 (“McLaughlin 2016”).
McLaughlin, T., et al., “Using the Neuroadaptagen KB200z to Ameliorate Terrifying, Lucid Nightmares in RDS Patients: the Role of Enhanced, Brain-Reward, Functional Connectivity and Dopaminergic Homeostasis,” J Reward Defic Syndr. 2015; 1(1):24-35 (“McLaughlin I2015”).
McLaughlin, T., et al., “Putative dopamine agonist (KB220Z) attenuates lucid nightmares in PTSD patients: role of enhanced brain reward functional connectivity and homeostasis redeeming joy,” J Behav Addict., 2015; 4(2): 106-15 (“McLaughlin II 2015”).
McLaughlin, T., et al., “Hypothesizing repetitive paraphilia behavior of a medication refractive Tourette's syndrome patient having rapid clinical attenuation with KB220Z-nutrigenomic amino-acid therapy (NAAT),” J Behav Addict., 2013; 2(2):117-24 (“McLaughlin 2013”).
Mehta, et al., Brain Behav Immun, 2018; 74:133-142 (“Mehta 2018”).
Mestrovic, et al., Neurosci Letters, 2018; 666:38-43 (“Mestrovic 2018”).
Minichino, et al. Schizophrenia Res., 2017; 185:114-121 (“Minichino 2017”).
Morey, et al., BMC Psychiatry, 2011; 5; 11:76 (“Morey 2011”).
Nelson, et al., Addict Biol., 2014; 19(4): 700-7 (“Nelson”).
Noble, E. P., et al., “Allelic association of the D2 dopamine receptor gene with cocaine dependence,” Drug Alcohol Depend., 1993; 33(3):271-85 (“Noble 1993”).
Noble, E. P., et al., “Allelic association of the D2 dopamine receptor gene with receptor-binding characteristics in alcoholism, Arch Gen Psychiatry, 1991; 48(7):648-54 (“Noble 1991”).
O'Doherty, D. C. M., et al., “Frontal and subcortical grey matter reductions in PTSD,” Psychiatry Research Neuroimaging, 2017; 266:1-9 (“O'Doherty 2017”).
O'Doherty, D. C., et al., “A systematic review and meta-analysis of magnetic resonance imaging measurement of structural volumes in posttraumatic stress disorder,” Psychiatry Research, 2015; 232(1):1-33 (“O'Doherty 2015”).
Oporto, et al., Clin Oral Investigation, 2018; 22(1):331-337 (“Oporto 2018”).
Philip, N. S., et al., “Developing neuroimaging phenotypes of the default mode network in PTSD: integrating the resting state, working memory, and structural connectivity,” Journal of Visualized Experiments: JoVE, 2014(89) (“Philip 2014”).
Prasad, et al., BMC Res. Notes, 2013; 6:418 (“Prasad 2013”).
Raj an, et al., Mov Disord Clin Pract, 2018; 5(4): 413-416 (“Rajan 2018”).
Reed, et al., Plosone, 2018; 13(4): eo195189 (“Reed”).
Rouillard, C., et al., “Behavioral and biochemical evidence for a different effect of repeated administration of L-dopa and bromocriptine on denervated versus non-denervated striatal dopamine receptors, Neuropharmacology, 1987; 26 (11): 1601-6 (“Rouillard 1987”).
Roy-Byrne, P., et al., “Persistence and change of PTSD symptomatology—a longitudinal co-twin control analysis of the Vietnam Era Twin Registry,” Social Psychiatry and Psychiatric Epidemiology, 2004; 39(9):681-5 (“Roy-Byrne 2004”).
Saunders, E. C., et al., “The Prevalence of Posttraumatic Stress Disorder Symptoms among Addiction Treatment Patients with Cocaine Use Disorders,” J Psychoactive Drugs, 2015; 47(1):42-50 (“Saunders 2015”).
Segman, et al., Mol Psychiatry, 2002; 7(8):903-7 (“Segman 2002”).
Sheppard, C. W., et al., “The changing face of heroin addiction in the Haight-Ashbury,” Int J Addict. 1972; 7(1):109-22 (“Sheppard 1972”).
Slavich, et al., Psychoneuroendocrinology, 2014; 49:141-9 (“Slavich 2014”).
Smith, B. H., et al., “Substance misuse of gabapentin,” The British Journal of General Practice: the journal of the Royal College of General Practitioners, 2012; 62(601):406-7 (“Brown 2012”).
Starkman, B. G., et al., “Epigenetics-beyond the genome in alcoholism,” Alcohol Res., 2012; 34(3):293-305 (“Starkman 2012”).
Szutorisz, H., et al., “Parental THC exposure leads to compulsive heroin-seeking and altered striatal synaptic plasticity in the subsequent generation,” Neuropsychopharmacology: official publication of the American College of Neuropsychopharmacology, 2014; 39(6):1315-23 (“Szutorisz 2014”).
Thanos, P. K., “Dopamine D2 gene expression interacts with environmental enrichment to impact lifespan and behavior,” Oncotarget., 2016 (“Thanos 2016”).
Thanos, P. K., et al., “D2R DNA transfer into the nucleus accumbens attenuates cocaine self-administration in rats,” Synapse (New York, N.Y.), 2008; 62(7):481-6 (“Thanos 2008”).
Thanos, P. K., et al., “Dopamine D2R DNA transfer in dopamine D2 receptor-deficient mice: effects on ethanol drinking,” Life Sci., 2005; 77(2):130-9 (“Thanos 2005”).
Tian, et al., Biol Res Nurs, 2015; 17(4):364-72 (“Tian 2015”).
Tsang, et al., NPJ Schizophrenia, 2015; 1:14002 (“Tsang 2015”).
van Wingen, G. A., et al., “Persistent and reversible consequences of combat stress on the mesofrontal circuit and cognition,” Proc Natl Acad Sci USA., 2012; 109(38):15508-13 (“van Wingen 2012”).
Vaswani, K. K., et al., “Cold swim stress-induced changes in the levels of opioid peptides in the rat CNS and peripheral tissues,” Pharmacol Biochem Behav., 1988; 29(1):163-8 (“Vaswani 1988”).
Valente, et al., J Mol Neurosci, 2011; 44(1):59 (“Valente 2011”).
Verhoeven, et al., Brain Behav, 2012; 2(6):806-13 (“Verhoeven 2012”).
Voisey, et al., Depress Anxiety, 2009; 26(1):28-33 (“Voisey 2009”).
Walsh, et al., J Affect Disord., 2014; 161:91-6 (“Walsh 2014”).
Winkler, et al., J Clin Neurosci; 2017; 35:109-116 (“Winkler 2017”).
Xie, et al., Am J Med Genet B Neuropsychiatric Genet, 2012; 159B(6):644-52 (“Xie 2012”).
Zainal, et al., BMC Neurol., 2015; 15:59 (“Zainal 2015”).
Zhang, K., et al., “A DRD2/ANNK1-COMT Interaction, Consisting of Functional Variants, Confers Risk of Post-traumatic Stress Disorder in Traumatized Chinese,” Frontiers in psychiatry, 2018; 9:170 (“Zhang 2018”).
Zhang, et al., Fron Behav Neurosci, 2017; 1; 11:17 (“Zhang 2017”).
Zhao, et al., Neuropsychobiology, 2016; 74(1):32-40 (“Zhao 2016”).
Zhu, et al., Plosone, 2013; 8; (8) (“Zhu 2013”).

What is claimed is:

1. A method comprising:
(a) obtaining a biological sample from each of a plurality of subjects;
(b) for each biological sample obtained from the plurality of subjects, performing an allelic analysis on the biological sample to detect the presence of a plurality of pre-determined alleles in the biological sample to identify the severity of a genetic addictive risk for a post-traumatic stress disorder (PTSD);
(c) identifying a first portion of the subjects in the plurality of subjects that have a high genetic addictive risk of PTSD based upon the allelic analysis of the biological samples;
(d) administering treatment to subjects in the first portion of subjects, wherein the treatment comprises:
(i) administering neuro-nutrients for dopamine regulation; and
(ii) administering precision behavioral management directed to achieve dopamine homeostasis.

2. The method of claim 1, wherein the precision behavioral management is selected from a group consisting of talk therapies, lifestyle measures that promote natural endorphin release, and support groups and systems.

3. The method of claim 2, wherein the lifestyle measures that promote natural endorphin release are selected from a group consisting of diet, mediation, and yoga.

4. The method of claim 1, wherein the plurality of subjects comprise people selected from a group of persons consisting of persons who recently enlisted in the military, persons who are enlisting in the military, persons who are trying to enlist in the military, and combinations thereof.

5. The method of claim 4, wherein the subjects in the first portion of subjects receive waivers from the military based upon them receiving the treatment.

6. The method of claim 4, wherein subjects in the first portion of subjects are assigned non-combat duties in the military based upon the subjects in the first portion of subjects having a high genetic addictive risk of PTSD.

7. The method of claim 1, wherein the plurality of subjects comprise individuals that have already been diagnosed with PTSD.

8. The method of claim 7, wherein the individuals comprises current and/or former military personnel.

9. The method of claim 7, wherein the administering treatments are varied among the subjects in the first portion of subjects based upon the allelic analysis on the biological sample for the subject.

10. The method of claim 7 further comprising:
(a) identifying a second portion of the subjects in the plurality of subjects that do not have a high genetic addictive risk of PTDS; and
(b) administering treatment to subjects to the second portion of subjects, wherein the treatments administered to the first and second portion of subjects are varied among the subjects in the first and second portion of subjects based upon the allelic analysis on the biological sample for the subject.

11. The method of claim 1, wherein the neuro-nutrients are comprised of precursor amino acids.

12. The method of claim 1, wherein the neuro-nutrients are dopamine regulators.

13. The method of claim 12, wherein the neuro-nutrients are regulators for glutamate, dopamine, serotonin, and endorphins.

14. The method of claim 1, wherein the method further comprises epigenetic testing of at least some of the plurality of subjects.

15. The method of claim 1, where the method further comprises epigenetic testing of at least some of the first portion of the subjects.

16. The method of claim 15, wherein the administration treatment to subjects in the first portion of subjects is varied based upon the epigenetic testing of the subject.

17. The method of claim 15, wherein the epigenetic testing comprises testing regarding environmental insults and/or abuse during childhood of the subject.

18. The method of claim 1 further comprising
(a) identifying a first sub-portion of the first portion of subjects that have a high addictive risk of alcohol and/or substance abuse based upon the allelic analysis of the biological samples; and
(b) varying the administering treatment of the first sub-portion based upon the upon the allelic analysis on the biological sample for the subject.

19. The method of claim 1, wherein
(a) for the pre-determined alleles being detected in the allelic analysis
(i) each of the alleles in the plurality of alleles is associated with a gene in a plurality of genes;
(ii) there are at least ten genes in the plurality of pre-determined genes; and
(iii) there is at least one allele for each of the genes in the plurality of pre-determined genes;
(b) the method further includes assigning a count for each of the alleles in the plurality of pre-determined alleles that was detected in the biological subject, wherein the count for the particular allele is the number of the particular allele detected to be present in the biological sample;
(c) the method further includes determining a risk score for the subject based upon the count, wherein the risk score is the sum of the counts; and
(d) the identification of the severity of the genetic addictive risk for PTSD is determined from the count.

20. The method of claim 19, wherein
(a) the risk score in a first pre-determined range identifies a low genetic addictive risk for PTSD; and
(b) the risk score in a second pre-determined range identifies a high genetic addictive risk for PTSD

21. The method of claim 20, wherein the second pre-determined range is 4 and above.

22. The method of claim 21, wherein the first pre-determined range is 0 or 1.

23. The method of claim 21, wherein the second pre-determined range is 7 or above.

24. The method of claim 21, wherein the first pre-determined range is 3 or below.

25. The method of claim 19, wherein at least five alleles in the plurality of the pre-determined alleles comprise
(a) allele G of gene DRD1;
(b) allele A1 of gene DRD2;
(c) allele S or L of gene HTTLPR;
(d) allele G of gene COMT; and
(e) allele G of gene OPRM1.

26. The method of claim 19, wherein at least eight alleles in the plurality of the pre-determined alleles is selected from a group consisting of:
(a) allele G of gene DRD1;
(b) allele A1 of gene DRD2;
(c) allele C of gene DRD3;
(d) allele C of gene DRD4;
(e) allele 9R of gene DAT1;
(f) allele 7-11R of gene DRD4;
(g) allele S or L of gene HTTLPR;
(h) allele 4R of gene MAOA;
(i) allele G of gene COMT;
(j) allele G of gene OPRM1; and
(k) allele 181 of gene GABRB3.

27. The method of claim 19, wherein the plurality of the pre-determined alleles comprises:
(a) allele G of gene DRD1;
(b) allele A1 of gene DRD2;
(c) allele C of gene DRD3;
(d) allele C of gene DRD4;
(e) allele 9R of gene DAT1;
(f) allele 7-11R of gene DRD4;
(g) allele S or L of gene HTTLPR;
(h) allele 4R of gene MAOA;
(i) allele G of gene COMT;
(j) allele G of gene OPRM1; and
(k) allele 181 of gene GABRB3.

28. The method of claim 19, wherein the plurality of the pre-determined alleles consist of:
(a) allele G of gene DRD1;
(b) allele A1 of gene DRD2;
(c) allele C of gene DRD3;
(d) allele C of gene DRD4;
(e) allele 9R of gene DAT1;
(f) allele 7-11R of gene DRD4;
(g) allele S or L of gene HTTLPR;
(h) allele 4R of gene MAOA;
(i) allele G of gene COMT;
(j) allele G of gene OPRM1; and
(k) allele 181 of gene GABRB3.
